(19) United States
(12) Patent Application Publication
(10) Pub. No.: US 2024/0124496 A1
(43) Pub. Date: Apr. 18, 2024

(54) BORONIC ACID COMPOUND AND METHOD FOR PRODUCING SAME

(71) Applicant: OSAKA UNIVERSITY, Suita (JP)

(72) Inventors: Yoshifumi SHIRAKAMI, Suita (JP); Kazuko KANEDA, Suita (JP); Yuichiro KADONAGA, Suita (JP); Tadashi WATABE, Suita (JP); Atsushi TOYOSHIMA, Suita (JP); Koichi FUKASE, Suita (JP); Atsushi SHINOHARA, Suita (JP); Toshio YAMANAKA, Ami-machi, Inashiki-gun (JP); Yutaka KONDOH, Ami-machi, Inashiki-gun (JP)

(73) Assignee: OSAKA UNIVERSITY, Suita (JP)

(21) Appl. No.: 18/551,674

(22) PCT Filed: Mar. 24, 2022
(86) PCT No.: PCT/JP2022/013808

§ 371 Date: Sep. 21, 2023

(30) Foreign Application Priority Data
Mar. 25, 2021 (JP) 2021-052352

(51) Int. Cl. C07F 5/02; B01J 31/22; B01J 31/24; C07B 59/00; C07C 227/16; C07F 13/00

(52) U.S. Cl. CPC C07F 5/025; B01J 31/2295; B01J 31/2409; C07B 59/00; C07C 227/16; C07F 13/00; B01J 2231/40; B01J 2531/004; B01J 2531/824; B01J 2531/842; C07B 2200/05

(57) ABSTRACT

The invention provides a method for producing radiolabeled tyrosine derivatives with good purity and stability, by a safe method suitable for industrial production of pharmaceuticals. The invention relates to a method for producing Compound (5) and Radiolabeled Compound (6) as follows:

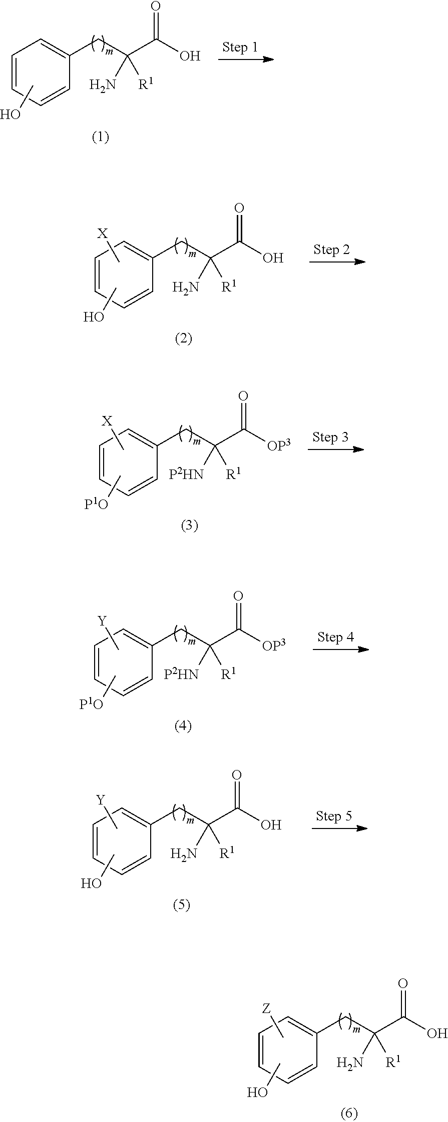

wherein each symbol is as defined in the description.

### TECHNICAL FIELD

The present invention relates to novel boronic acid compounds which are intermediates for producing radiolabeled tyrosine derivatives useful as anti-cancer drugs, and methods zo for producing the same, and methods for producing radiolabeled tyrosine derivatives using the same.

### BACKGROUND ART

Radiolabeled tyrosine derivatives such as astato(²¹¹At)-α-methyl-L-tyrosine (²¹¹At-AAMT) are drugs that are taken up into tumor cells via LAT1 amino acid transporter specifically expressed in tumors, and are expected to be useful as anti-cancer drugs (Patent Document 1).

Patent Document 1 discloses that ²¹¹At-AAMT is conventionally produced by dissolving α-methyl-L-tyrosine (AMT) in sulfuric acid, adding mercury sulfate to the solution to introduce mercury on the benzene ring, and then subjecting the resulting compound to an astatine exchange reaction (hereinafter to be also referred to as the mercury method). Since this method requires use of mercury, which is a hazardous substance, it cannot be said to be a method suitable for the production of pharmaceuticals in terms of safety. In addition, the above method is not suitable for industrial production because the synthetic yield varies relatively greatly between production batches. Furthermore, iodine-substituted compounds and halogen-disubstituted compounds are produced as by-products, which poses a problem in terms of purity. It has also been reported that the produced ²¹¹At-AAMT is unstable (Non-Patent Document 1).

In addition, it is generally known that halogen is easily introduced into the 3-position of a tyrosine derivative by halogenation in the presence of an oxidant. However, this method is not effective only for astatine (Non-Patent Document 2).

On the other hand, the present inventors have already reported that a boryl group (—B(OH)₂) introduced into an aryl group has excellent astatine-substituting ability (Patent Document 2).

### DOCUMENT LIST

### Patent Document

[Patent Document 1] WO 2019/176505

[Patent Document 2] WO 2019/027059

### Non-Patent Document

[Non-Patent Document 1] J Surg Oncol 1988; 37:192-7

[Non-Patent Document 2] Int J Appl Radiat Isotop 1979; 30:749-52

### SUMMARY OF THE INVENTION

### Problems to be Solved by the Invention

The present invention aims to provide a method for producing radiolabeled tyrosine derivatives with good purity and stability, by a safe method suitable for industrial production of pharmaceuticals.

### Means of Solving the Problems

The present inventors have focused on the production method described in Patent Document 2 as a production method that does not require use of hazardous substances such as mercury, and have conducted intensive studies in an attempt to produce tyrosine boronic acid derivatives or its esters as raw materials for the method, which resulted in the completion of the present invention.

Accordingly, the present invention provides the following.

[1] A method for producing a compound represented by Formula (5) or a salt thereof (hereinafter to be also referred to as Compound (5)), comprising the following Steps 1 to 4;

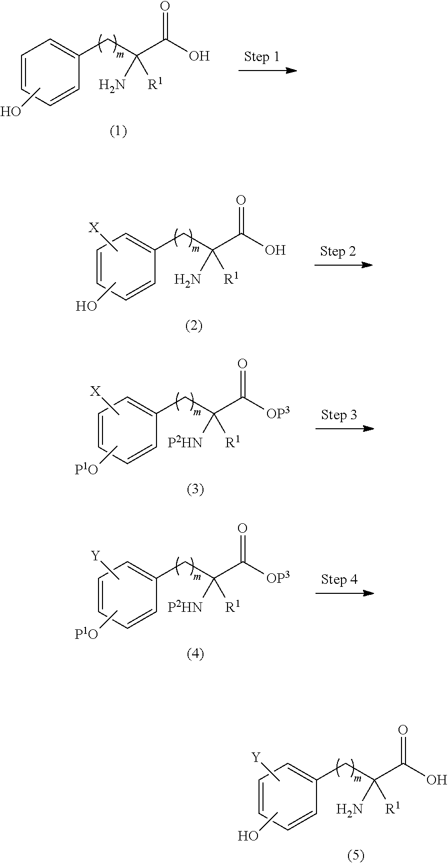

wherein
R¹ is a hydrogen atom or a C₁₋₄ alkyl group;
P¹ is an ether-type hydroxy-protecting group;
P² is an amino-protecting group;
P³ is a carboxy-protecting group;
m is 0, 1 or 2;
X is a halogen atom; and
Y is a boryl group (—B(OH)₂) or its ester group,
Step 1: a step of halogenating a compound represented by Formula (1) or a salt thereof (hereinafter to be also referred to as Compound (1)) to obtain a compound represented by Formula (2) or a salt thereof (hereinafter to be also referred to as Compound (2));
Step 2: a step of protecting the amino group and carboxy group of the compound represented by Formula (2) or a salt thereof and protecting the hydroxy group of the compound or salt with an ether-type protecting group to obtain a compound represented by Formula (3) (hereinafter to be also referred to as Compound (3));
Step 3: a step of reacting the compound represented by Formula (3) with a reagent for introducing boronic acid in the presence of a palladium catalyst and a base to obtain a compound represented by Formula (4) (hereinafter to be also referred to as Compound (4)); and
Step 4: a step of removing the protecting groups for the carboxy group, amino group and hydroxy group of the compound represented by Formula (4) to obtain the compound represented by Formula (5) or a salt thereof.

[2] The method of the above-mentioned [1], wherein P¹ is a benzyl group or a p-methoxybenzyl group.

[3] The method of the above-mentioned [1] or [2], wherein P³ is a benzyl group or a C₁₋₂ alkyl group.

[4] The method of the above-mentioned [1], wherein P¹ and P³ are both benzyl groups.

[5] The method of any one of the above-mentioned [1] to [4], wherein the bonding position of the hydroxy group on the benzene ring in Formula (5) is the 4-position or 3-position.

[6] The method of the above-mentioned [5], wherein the bonding positions of the hydroxy group and the group −Y on the benzene ring in Formula (5) are adjacent to each other.

[7] The method of any one of the above-mentioned [1] to [4], wherein, in Formula (5), the bonding position of the hydroxy group on the benzene ring is the 4-position, and the bonding position of the group −Y on the benzene ring is the 3-position.

[8] The method of any one of the above-mentioned [1] to [7], wherein R¹ is a hydrogen atom or a methyl group.

[9] The method of any one of the above-mentioned [1] to [8], wherein the palladium catalyst to be used in Step 3 is [1,1′-bis(diphenylphosphino)ferrocene]dichloropalladium(II) (PdCl₂(dppf)).

[10] The method of the above-mentioned [9], wherein the reaction of Step 3 is carried out in a sulfoxide solvent or an amide solvent.

[11] The method of the above-mentioned [9], wherein the base to

be used in Step 3 is an alkali metal acetate.

[12] The method of any one of the above-mentioned [1] to [11], wherein Y is a boryl group (—B(OH)₂) or a 4,4,5,5-tetramethyl-1,3,2-dioxaboran-2-yl group.

[13] A method for producing a compound represented by Formula (5) or a salt thereof, comprising the following Step 4;

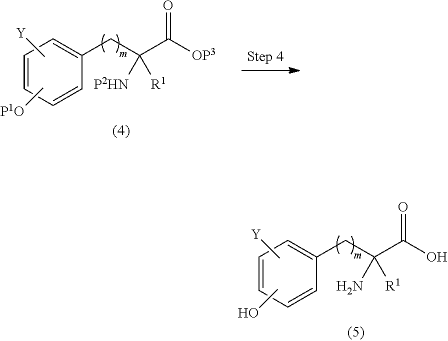

wherein
R¹ is a hydrogen atom or a C₁₋₄ alkyl group;
P¹ is an ether-type hydroxy-protecting group;
P² is an amino-protecting group;
P³ is a carboxy-protecting group;
m is 0, 1 or 2; and
Y is a boryl group (—B(OH)₂) or its ester group,
Step 4: a step of removing the protecting groups for the carboxy group, amino group and hydroxy group of a compound represented by Formula (4) to obtain the compound represented by Formula (5) or a salt thereof.

[14] A method for producing a compound represented by Formula (4), comprising the following Step 3;

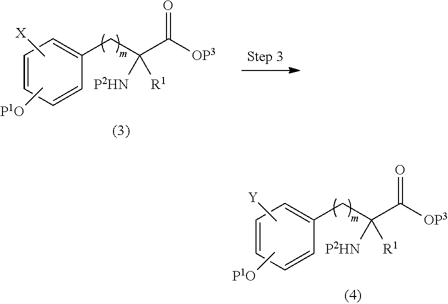

wherein
R¹ is a hydrogen atom or a C₁₋₄ alkyl group;
P¹ is an ether-type hydroxy-protecting group;
P² is an amino-protecting group;
P³ is a carboxy-protecting group;
m is 0, 1 or 2;
X is a halogen atom; and
Y is a boryl group (—B(OH)₂) or its ester group,
Step 3: a step of reacting a compound represented by Formula (3) with a reagent for introducing boronic acid in the presence of a palladium catalyst and a base to obtain the compound represented by Formula (4).

[15] A compound represented by the following Formula (5a) or a salt thereof (hereinafter to be also referred to as Compound (5a));

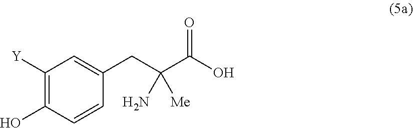

wherein Y is a boryl group (—B(OH)₂) or its ester group.

A compound represented by the following Formula (4a) (hereinafter to be also referred to as Compound (4a));

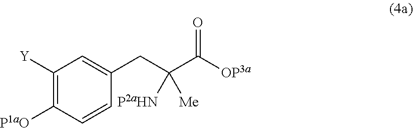

wherein
P^{1a} is a benzyl group or a p-methoxybenzyl group;
P^{2a} is a tert-butoxycarbonyl group;
P^{3a} is a benzyl group or a C₁₋₂ alkyl group; and
Y is a boryl group (—B(OH)₂) or its ester group.

[17] A method for producing a radiolabeled compound represented

by Formula (6) or a salt thereof (hereinafter to be also referred to as Radiolabeled Compound (6)), comprising the following Step 5;

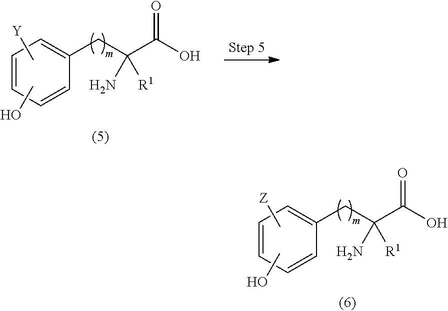

wherein
R¹ is a hydrogen atom or a C₁₋₄ alkyl group;
m is 0, 1 or 2;
Y is a boryl group (—B(OH)₂) or its ester group; and
Z is ²¹¹At, ²¹⁰At, ¹²³I, ¹²⁴I, ¹²⁵I or ¹³¹I,
Step 5: a step of reacting a compound represented by Formula (5) or a salt thereof with a radionuclide selected from ²¹¹At, ²¹⁰At, ¹²³I, ¹²⁴I, ¹²⁵I and ¹³¹I, in the presence of a reagent selected from an alkali metal iodide, an alkali metal bromide, N-bromosuccinimide, N-chlorosuccinimide, N-iodosuccinimide and hydrogen peroxide, in water to obtain the radiolabeled compound represented by Formula (6) or a salt thereof.

[18] The method of the above-mentioned [17], wherein the compound represented by Formula (5) or a salt thereof is produced by a method as defined in any one of the above-mentioned [1] to [12].

[19] The method of the above-mentioned [17] or [18], wherein the reaction is carried out in an organic solvent-free system.

[20] The method of any one of the above-mentioned [17] to [19], wherein the reaction is carried out within the range of room temperature to 100° C.

[21] The method of any one of the above-mentioned [17] to [20], wherein the radionuclide is ²¹¹At or ¹³¹I, and the reagent is selected from potassium iodide and N-bromosuccinimide.

[22] The method of any one of the above-mentioned [17] to [21], further comprising a step of purifying the radiolabeled compound represented by Formula (6) or a salt thereof.

[23] The method of any one of the above-mentioned [17] to [22], further comprising a step of stabilizing the radiolabeled compound represented by Formula (6) or a salt thereof by adding ascorbic acid.

### Effect of the Invention

According to the present invention, tyrosine derivatives into which a boryl group (—B(OH)₂) or its ester group is introduced can be obtained, and therefore, the use of the compounds makes it possible to produce radiolabeled tyrosine derivatives with good purity and stability, by a safe method suitable for industrial production of pharmaceuticals without using hazardous substances.

### BRIEF DESCRIPTION OF THE DRAWINGS

FIG. 1 shows the HPLC chart of 3-(4,4,5,5-tetramethyl-1,3,2-dioxaboran-2-yl)-α-methyl-L-tyrosine hydrochloride (5) obtained in Example 1.

FIG. 2 shows the HPLC chart of 3-borono-α-methyl-L-tyrosine hydrochloride (5) obtained in Example 2.

FIG. 3 shows the results of thin-layer chromatography (TLC) analysis of the reaction solution prepared in Example 3.

FIG. 4 shows the results of thin-layer chromatography (TLC) analysis of the reaction solution prepared in Example 4.

FIG. 5 shows the results of thin-layer chromatography (TLC) analysis of the reaction solution prepared in Example 5.

FIG. 6a shows the ²¹¹At-AAMT intracellular uptake (synthesized by the method of the present invention), and FIG. 6b shows the ²¹¹At-AAMT intracellular uptake (synthesized by the mercury method).

FIG. 7 shows cell viability after addition of ²¹¹At-AAMT.

### DESCRIPTION OF EMBODIMENTS

The present invention is explained in detail in the following.

In the present specification, examples of the “halogen atom” include a fluorine atom, a chlorine atom, a bromine atom and an iodine atom.

In the present specification, examples of the “C₁₋₄ alkyl group” include methyl, ethyl, propyl, isopropyl, butyl, isobutyl, sec-butyl and tert-butyl.

In the present specification, examples of the “C₁₋₆ alkyl group” include methyl, ethyl, propyl, isopropyl, butyl, isobutyl, sec-butyl, tert-butyl, pentyl, isopentyl, neo-pentyl, 1-ethylpropyl, hexyl, isohexyl, 1,1-dimethylbutyl, 2,2-dimethylbutyl, 3,3-dimethylbutyl and 2-ethylbutyl.

In the present specification, examples of the “ether-type hydroxy-protecting group” include a benzyl group, a p-methoxybenzyl group, a methoxymethyl group, a trimethylsilyl group, a triethylsilyl group, a trityl group, a tert-butyldimethylsilyl group, a tetrahydropyranyl group and the like. Among them, benzyl ether-type hydroxy-protecting groups such as a benzyl group and a p-methoxybenzyl group are preferred.

In the present specification, examples of the “amino-protecting group” include a tert-butoxycarbonyloxy group, a benzyloxycarbonyl group, a 9-fluorenylmethyloxycarbonyl group and the like.

In the present specification, examples of the “carboxy-protecting group” include a benzyl group, a C₁₋₂ alkyl group (a methyl group, an ethyl group), a tert-butyl group and the like.

In the present specification, the term “boryl group (—B(OH)₂)” is also referred to as a dihydroxyboryl group.

In the present specification, examples of the “ester group of boryl group” include the following groups.

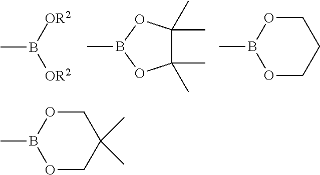

wherein R² is a C₁₋₆ alkyl group.
R¹ is preferably a hydrogen atom or a methyl group, more preferably a methyl group.
P¹ is preferably a benzyl ether-type hydroxy-protecting group, more preferably a benzyl group or a p-methoxybenzyl group, particularly preferably a benzyl group.
P² is preferably a tert-butoxycarbonyloxy group or a benzyloxycarbonyl group, more preferably a tert-butoxycarbonyloxy group.
P³ is preferably a benzyl group or a C₁₋₂ alkyl group (a methyl group, an ethyl group), more preferably a benzyl group.
m is preferably 1.
X is preferably an iodine atom or a bromine atom, particularly preferably an iodine atom.
Y is preferably a boryl group (—B(OH)₂) or a 4,4,5,5-tetramethyl-1,3,2-dioxaboran-2-yl group.

In the present specification, when Compound (1), Compound (2), Compound (5) and Radiolabeled Compound (6) are each a salt, examples of such salt include metal salts (e.g., alkali metal salts such as sodium salt and potassium salt; alkaline-earth metal salts such as calcium salt, magnesium salt and barium salt), ammonium salts, salts with organic bases (e.g., trimethylamine, triethylamine, pyridine, picoline, 2,6-lutidine), salts with inorganic acids (e.g., hydrochloric acid, hydrobromic acid, nitric acid, sulfuric acid), salts with organic acids (e.g., formic acid, acetic acid, trifluoroacetic acid, phthalic acid, fumaric acid, oxalic acid, tartaric acid, maleic acid, citric acid, succinic acid, malic acid), and the like.

In the present invention, a method for producing Compound (5) comprises the following Steps 1 to 4.

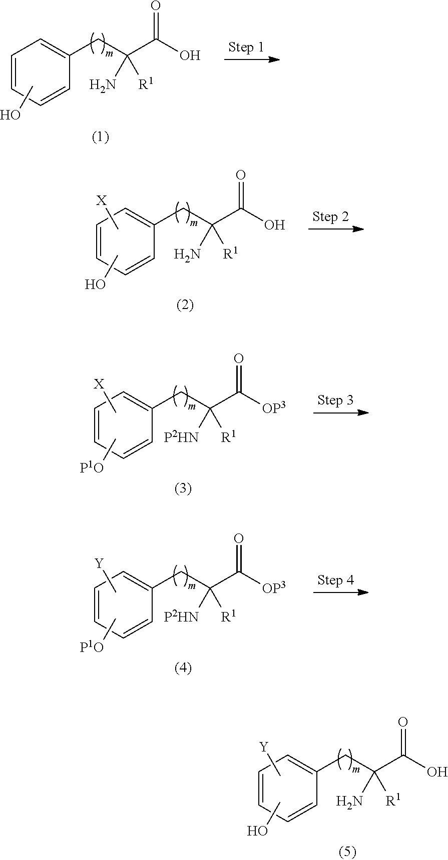

Step 1 is a step of halogenating Compound (1) to obtain Compound (2).

The halogenation can be carried out by reacting Compound (1) with a halogenating agent.

Examples of Compound (1) include tyrosine, α-methyltyrosine, m-tyrosine, α-methyl-m-tyrosine and the like. Compound (1) may be L-form, D-form or DL-form. Among them, tyrosine and α-methyltyrosine are preferably used, and α-methyltyrosine is particularly preferably used. Compound (1) may be a commercially available product.

The halogenating agent to be used is preferably an iodinating agent or a brominating agent, particularly preferably an iodinating agent.

Examples of the iodinating agent include iodine, N-iodosuccinimide and the like.

Examples of the brominating agent include bromine, N- bromosuccinimide and the like.

The amount of the halogenating agent to be used is generally 1 to 5 mol, preferably 1 to 2 mol, per 1 mol of Compound (1).

When the halogenating agent is iodine, the reaction is carried out in the presence of potassium iodide and conc. ammonia (28%). The amount of the potassium iodide to be used is generally 0.5 to 5 mol, preferably 1 to 2 mol, per 1 mol of the iodine, and the amount of the conc. ammonia to be used is generally 5 to 200 mol, preferably 20 to 50 mol, per 1 mol of the iodine.

The reaction is carried out generally in a solvent. The solvent to be used in the present invention is not particularly limited as long as it does not adversely affect the reaction. Examples thereof include water; alcohol solvents such as ethanol, methanol and isopropanol; halogen solvents such as carbon tetrachloride, and the like. These may be used in combination of two or more. Among them, water is preferably used.

The amount of the solvent to be used is generally 0.1 to 100-fold in volume relative to Compound (1).

When the halogenating agent is iodine, the reaction is carried out preferably, for example, by adding (preferably dropwise) a mixture of Compound (1) and conc. ammonia (and solvent if necessary) to a mixture of iodine, potassium iodide and solvent.

The reaction is carried out generally within the range of −100 to 20° C., preferably −20 to 10° C. The reaction time varies depending on the reaction temperature, and it is generally about 30 min to about 24 hr, preferably about 1 to about 12 hr.

The completion of the reaction can be confirmed by thin-layer chromatography, liquid chromatography and the like.

After the completion of the reaction, Compound (2) can be isolated and/or purified from the reaction mixture by separation means such as concentration, crystallization, recrystallization, distillation, solvent extraction, fractional distillation and chromatography, according to a conventional method.

By this reaction, a halogen is introduced into the 3-position of the benzene ring in the case of tyrosine and α-methyltyrosine (both of which have a hydroxy group at the 4-position of the benzene ring), and into the 4-position or 6-position of the benzene ring in the case of m-tyrosine and α-methyl-m-tyrosine (both of which have a hydroxy group at the 3-position of the benzene ring).

Step 2 is a step of protecting the amino group and carboxy group of Compound (2), and protecting the hydroxy group with an ether-type protecting group to obtain Compound (3).

The present inventors initially attempted to introduce a boryl group or its ester group into Compound (2) by utilizing a well-known reaction of a halogen compound with a reagent for introducing boronic acid. However, it was unexpectedly found that the reaction hardly proceeded. Predicting that the hydroxy group on the benzene ring might be the cause, they attempted to protect the hydroxy group with various protective groups before introducing the boryl group or its ester group.

As a result, it was unexpectedly found that, depending on the type of protecting group, the introduction reaction of the boryl group or its ester group may be difficult to proceed.

The present inventors have conducted intensive studies on hydroxy-protecting groups in order to solve such hitherto unknown problems specific to tyrosine derivatives, that is, specific to benzene rings having a hydroxy group, and found for the first time that, by adopting a specific protecting group (an ether-type protecting group, especially a benzyl ether-type protecting group), a boryl group or its ester group can be introduced into the benzene ring. Thus, in the present invention, the selection of the hydroxy-protecting group for Compound (2) is an important key in the introduction of a boryl group or its ester group.

In the present invention, the hydroxy-protecting group is an ether-type protecting group (P¹). As long as the hydroxy-protecting group is an ether-type protecting group (P¹), a boryl group or its ester group can be introduced into the benzene ring. On the other hand, when the hydroxy-protecting group is an ester-type protecting group such as an acetyl group, a benzoyl group and a pivaloyl group, the introduction reaction of a boryl group or its ester group hardly proceeds.

In terms of the introduction of a boryl group or its ester group in a good yield, the ether-type hydroxy-protecting group (P¹) is preferably a benzyl ether-type protecting group, more preferably a benzyl group or a p-methoxybenzyl group, particularly preferably a benzyl group. Since a benzyl group and a p-methoxybenzyl group have not much steric hinderance, and thus a boryl group or its ester group can be introduced in a high yield.

The present inventors also found that the introduced boryl group or its ester group may be unexpectedly eliminated under conditions of strong acids such as highly concentrated hydrochloric acid, nitric acid, sulfuric acid, boron tribromide and boron trifluoride in the deprotection step after introduction. Such elimination is usually difficult to occur in benzene rings having no hydroxy group, and is a problem specific to benzene rings having a hydroxy group.

The present inventors have further conducted intensive studies on hydroxy-protecting groups in order to solve such hitherto unknown problems specific to tyrosine derivatives, that is, specific to benzene rings having a hydroxy group, and found for the first time that, by adopting a benzyl ether-type protecting group (particularly a benzyl group or a p-methoxybenzyl group), the subsequent deprotection step can be carried out under milder conditions (e.g., catalytic hydrogenation). A p-methoxybenzyl group can also be removed with an oxidant such as 2,3-dichloro-5,6-dicyano-p-benzoquinone, trifluoroacetic acid, and relatively low-concentrated hydrochloric acid. The present inventors also found that, by adopting such deprotection method, the elimination of the boryl group or its ester group is difficult to occur.

Thus, in the present invention, the selection of the hydroxy-protecting group for Compound (2) is an important key in the elimination of the boryl group or its ester group.

Moreover, the protecting groups (P², P³) for the amino group and carboxy group are also selected from those that can be removed under conditions that hardly cause the elimination of the boryl group and its ester group in Step 4.

The amino-protecting group (P²) is preferably a tert-butoxycarbonyloxy group.

The carboxy-protecting group (P³) is preferably a benzyl group or a C₁₋₂ alkyl group (a methyl group, an ethyl group), particularly preferably a benzyl group.

The preferred combinations include
a combination in which the ether-type hydroxy-protecting group (P¹) is a benzyl group, the amino-protecting group (P²) is a tert-butoxycarbonyloxy group, and the carboxy-protecting group (P³) is a benzyl group, and
a combination in which the ether-type hydroxy-protecting group (P¹) is a p-methoxybenzyl group, the amino-protecting group (P²) is a tert-butoxycarbonyloxy group, and the carboxy-protecting group (P³) is a C₁₋₂ alkyl group (a methyl group, an ethyl group).

All of the above protective groups can be removed under mild conditions, and therefore, the elimination of the boryl group or its ester group is difficult to occur.

Each protective group can be introduced according to a method known per se, and the order of the introduction of each protective group is appropriately determined depending on the protective group.

After the completion of the reaction, Compound (3) can be isolated and/or purified from the reaction mixture by separation means such as concentration, crystallization, recrystallization, distillation, solvent extraction, fractional distillation and chromatography, according to a conventional method.

Step 3 is a step of reacting Compound (3) with a reagent for introducing boronic acid, in the presence of a palladium catalyst and a base to obtain Compound (4).

Examples of the reagent for introducing boronic acid include 4,4,4′,4′,5,5,5′,5′-octamethyl-2,2′-bi-1,3,2-dioxaborolane (also known as bis(pinacolato)diboron), 4,4,5,5-trimethyl-1,3,2-dioxaborolane, tetrahydroxydiborane and the like. Among them, bis(pinacolato)diboron is preferably used. These reagents for introducing boronic acid may be a commercially available product.

The amount of the reagent for introducing boronic acid to be used is generally 1 to 10 mol, preferably 1 to 3 mol, per 1 mol of Compound (3).

Examples of the palladium catalyst include [1,1′-bis(diphenylphosphino)ferrocene]dichloropalladium(II) (PdCl₂(dppf)) or its dichloromethane adduct, palladium acetate, tetrakis(triphenylphosphine)palladium(0) (Pd(PPh₃)₄) and the like. Among them, PdCl₂(dppf) is preferably used.

The amount of the palladium catalyst to be used is generally 0.001 to 1 mol, preferably 0.01 to 0.2 mol, per 1 mol of Compound (3).

Examples of the base include alkali metal acetates such as potassium acetate and sodium acetate, and the like. Among them, alkali metal acetates are preferably used, and potassium acetate is particularly preferably used.

The amount of the base to be used is generally 0.5 to 10 mol, preferably 1 to 5 mol, per 1 mol of Compound (3).

The reaction is carried out generally in a solvent. The solvent to be used in the present invention is not particularly limited as long as it does not adversely affect the reaction, and examples thereof include sulfoxide solvents such as dimethyl sulfoxide; amide solvents such as dimethylformamide, dimethylacetamide and N-methylpyrrolidinone; ether solvents such as tetrahydrofuran and 1,4-dioxane. These may be used in combination of two or more. Among them, sulfoxide solvents and amide solvents are preferably used, and dimethyl sulfoxide is particularly preferably used.

The amount of the solvent to be used is generally 0.1 to 100-fold in volume relative to Compound (3).

The reaction is carried out preferably, for example, by adding (preferably dropwise) a reagent for introducing boronic acid to a mixture of Compound (3), a palladium catalyst, a base and a solvent.

The reaction is carried out generally within the range of 0 to 200° C., preferably room temperature to 120° C. The reaction time varies depending on the reaction temperature, and it is generally about 10 min to about 48 hr, preferably about 1 to about 12 hr.

The completion of the reaction can be confirmed by thin-layer chromatography, liquid chromatography and the like.

After the completion of the reaction, Compound (4) can be isolated and/or purified from the reaction mixture by separation means such as concentration, crystallization, recrystallization, distillation, solvent extraction, fractional distillation, chromatography and the like, according to a conventional method.

Among Compound (4), a compound represented by the following Formula (4a) is a novel compound.

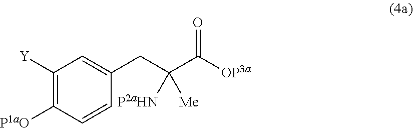

wherein
P^{1a} is a benzyl group or a p-methoxybenzyl group;
P^{2a} is a tert-butoxycarbonyl group;
P^{3a} is a benzyl group or a C₁₋₂ alkyl group; and
Y is as defined above.

Step 4 is a step of removing the protecting groups for the carboxy group, amino group and hydroxy group of Compound (4) to obtain Compound (5).

The removal of each protecting group is carried out according to a method known per se.

For example, when the hydroxy-protecting group (P¹)/carboxy-protecting group (P³) are each a benzyl group, the removal is carried out by catalytic hydrogenation.

When the hydroxy-protecting group (P¹) is a p-methoxybenzyl group, the removal is carried out by catalytic hydrogenation, or treatment with an acid such as trifluoroacetic acid and hydrogen chloride.

When the carboxy-protecting group (P³) is a C₁₋₂ alkyl group (a methyl group, an ethyl group), the removal is carried out by treatment with a base such as lithium hydroxide and sodium hydroxide.

When the amino-protecting group (P²) is a tert-butoxycarbonyl group, the removal is carried out by treatment with an acid such as trifluoroacetic acid and hydrogen chloride.

The above removals of the protecting groups can be carried out under mild conditions, and therefore, the elimination of the boryl group (—B(OH)₂) or its ester group is difficult to occur.

After the completion of the reaction, Compound (5) can be isolated and/or purified from the reaction mixture by separation means such as concentration, crystallization, recrystallization, distillation, solvent extraction, fractional distillation, chromatography and the like, according to a conventional method.

Among Compound (5), a compound represented by the following Formula (5a) or a salt thereof is a novel compound.

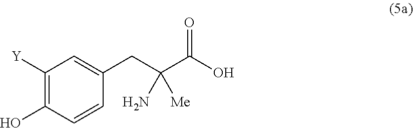

wherein Y is as defined above.

Compound (5) thus produced can be converted to Radiolabeled Compound (6) useful as an anti-cancer drug by a method including the following Step 5.

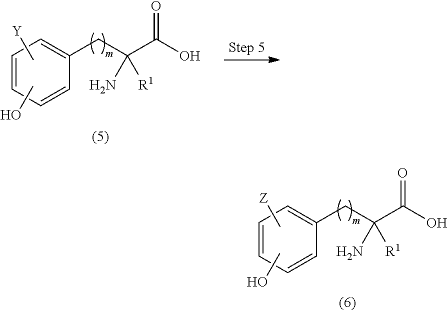

wherein Z is ²¹¹At, ²¹⁰At, ¹²³I, ¹²⁴I, ¹²⁵I or ¹³¹I, and the other symbols are as defined above.

Step 5 is a step of reacting Compound (5) with a radionuclide selected from ²¹¹At, ²¹⁰At, ¹²³I, ¹²⁴I, ¹²⁵I and ¹³¹I, in the presence of a reagent selected from an alkali metal iodide, an alkali metal bromide, N-bromosuccinimide, N-chlorosuccinimide, N-iodosuccinimide and hydrogen peroxide, in water to obtain Radiolabeled Compound (6).

Since the reaction in this step is carried out in water, Compound (5) may be in a free form or salt form as long as it can be dissolved in water. Examples of the salt form include a hydrochloride.

Examples of the alkali metal iodide include potassium iodide, sodium iodide and the like. Among them, potassium iodide is preferably used.

Examples of the alkali metal bromide include sodium bromide, potassium bromide and the like.

The preferred combinations of the radionuclide and the above reagent include
(1) a combination in which the radionuclide is ²¹¹At or ²¹⁰At, and the above reagent is selected from potassium iodide, sodium bromide, N-bromosuccinimide, N-chlorosuccinimide, N-iodosuccinimide and hydrogen peroxide; and
(2) a combination in which the radionuclide is ¹²³I, ¹²⁴I, ¹²⁵I or ¹³¹I, and the above reagent is selected from N-bromosuccinimide and N-chlorosuccinimide.

The above reagent may be used alone or in combination of two or more. The above reagent is used usually in the form of an aqueous solution.

As the preferred embodiment, the radionuclide is ²¹¹At or ¹³¹I, and the reagent is selected from potassium iodide and N-bromosuccinimide.

The more preferred embodiments include
an embodiment in which the radionuclide is ²¹¹At, and the reagent is potassium iodide, and
an embodiment in which the radionuclide is ¹³¹I, and the reagent is N-bromosuccinimide.

The above reagent is used in an amount sufficient to oxidize or reduce the radionuclide, and is used usually in a large excess amount relative to the radionuclide. It is used preferably in a concentration of 0.0001 to 0.2 mol/L, more preferably in a concentration of 0.001 to 0.1 mol/L, in terms of reaction efficiency and economic efficiency.

For the reaction, the radionuclide is used usually in the form of an aqueous solution. If necessary, an alkaline aqueous solution such as sodium hydroxide and buffer solution may be added to the aqueous solution in order to stabilize the radionuclide.

In the case of radionuclide ²¹¹At, first, ²¹¹At is produced by ²⁰⁹Bi(α,2n) ²¹¹At nuclear reaction resulting from the irradiation of bismuth with helium particles accelerated to 28 MeV by a cyclotron. Next, by heating, the target substance ²⁰⁹Bi is melted and the ²¹¹At is vaporized, and the vaporized ²¹¹At is collected in a liquid nitrogen trap, and dissolved in water to prepare an ²¹¹At stock solution. If necessary, an alkaline solution such as sodium hydroxide and buffer solution may be added thereto for the purpose of stabilizing ²¹¹At.

In the case of radionuclide ²¹⁰At, first, ²¹⁰At is produced by ²⁰⁹Bi (α, ³n) ²¹⁰At nuclear reaction resulting from the irradiation of bismuth with helium particles accelerated to 29 MeV or more by cyclotron. Next, by the same procedures as above, an aqueous ²¹⁰At solution is prepared.

In the case of radionuclide ¹²³I, it is available as an aqueous Na¹²³I solution.

In the case of radionuclide ¹²⁴I, first, ¹²⁴I is produced by ¹²⁴Te(p,n)¹²⁴I nuclear reaction resulting from the irradiation of tellurium with proton particles accelerated by cyclotron. Next, the target substance ¹²⁴Te is melted, and the ¹²⁴I is vaporized to prepare an aqueous ¹²⁴I sodium hydroxide solution.

In the case of radionuclide ¹²⁵I, it is available as an aqueous Na¹²⁵I solution.

In the case of radionuclide ¹³¹I, it is available as an aqueous Na¹³¹I solution.

²¹¹At has a half-life of 7.2 hours, ²¹⁰At has a half-life of 8.3 hours, and ¹²³I has a half-life of 13.2 hours. These radionuclides have a short half-life, and therefore, they should be used in the subsequent reaction immediately after the preparation. On the other hand, ¹²⁴I has a half-life of 4.2 days, ¹²⁵I has a half-life of 59.4 days, and ¹³¹I has a half-life of 8.04 days. Although these radionuclides have a relatively long half-life, they are preferably used in the subsequent reaction immediately after the preparation.

Compound (5) is used usually in a large excess amount relative to the radionuclide, preferably in a concentration of 0.0001 mol/l to 0.5 mol/l, more preferably in a concentration of 0.001 mol/l to 0.2 mol/1, per 1 Bq to 1,000 GBq of the radionuclide, in terms of reaction efficiency and economic efficiency.

The above reaction is carried out by mixing Compound (5), the above reagent and the radionuclide, and the mixing order is not particularly limited. The reaction is preferably carried out by adding an aqueous solution of the radionuclide followed by an aqueous solution of the above reagent to an aqueous solution of Compound (5), or by adding an aqueous solution of the above reagent followed by an aqueous solution of the radionuclide to an aqueous solution of Compound (5), more preferably by adding an aqueous solution of the radionuclide followed by an aqueous solution of the above reagent to an aqueous solution of Compound (5).

The above reaction is carried out in water, i.e., in an organic solvent-free system.

The above reaction is carried out at room temperature, specifically within the range of 0° C. to 40° C., preferably 10° C. to 35° C. In the production method of the present invention, the reaction proceeds rapidly even at room temperature, and is completed in a short time, for example, from 1 minute to 3 hours, especially from 1 min to 30 min.

The completion of the reaction is confirmed by the disappearance of the free radionuclide, using thin-layer chromatography (TLC) analysis.

In the production method of the present invention, Radiolabeled Compound (6) can be obtained in a high radiochemical yield of 60% or more, particularly 80% or more, especially 90% or more.

Since the reaction solution after the completion of the reaction contains neither an organic solvent nor a toxic reagent, the reaction solution can be immediately formulated into an injection and the like without isolating Radiolabeled Compound (6).

Compound (6) may be purified, if necessary, to remove by-products. This purification is preferably carried out by a solid-phase extraction column. As solid-phase extraction columns, those commonly used in the technical field can be used.

Furthermore, after the above purification, ascorbic acid or ascorbate may be added to a final concentration of 0.01% to 10%, preferably 0.1% to 5%. This makes it possible to suppress the decomposition of Compound (6) and retain it for a long period of time.

The reaction conditions such as solvent and reaction temperature in each step in the production method of the present invention described above are described in detail as representative examples in the examples below, but are not necessarily limited thereto, and those skilled in the art can make appropriate selections based on their general knowledge in organic synthesis.

### EXAMPLES

The present invention will be explained in detail by the following examples, which are merely examples and are not intended to limit the present invention and can be modified without departing from the scope of the present invention.

In the following Examples, the radiochemical yield was calculated using the following formula.

radiochemical yield (%)=(radioactivity of the desired compound on thin-layer plate/total radioactivity on thin-layer plate)×100

### Example 1

### Synthesis of 3-(4,4,5,5-tetramethyl-1,3,2-dioxaboran-2-yl)-α-methyl-L-tyrosine hydrochloride (5)

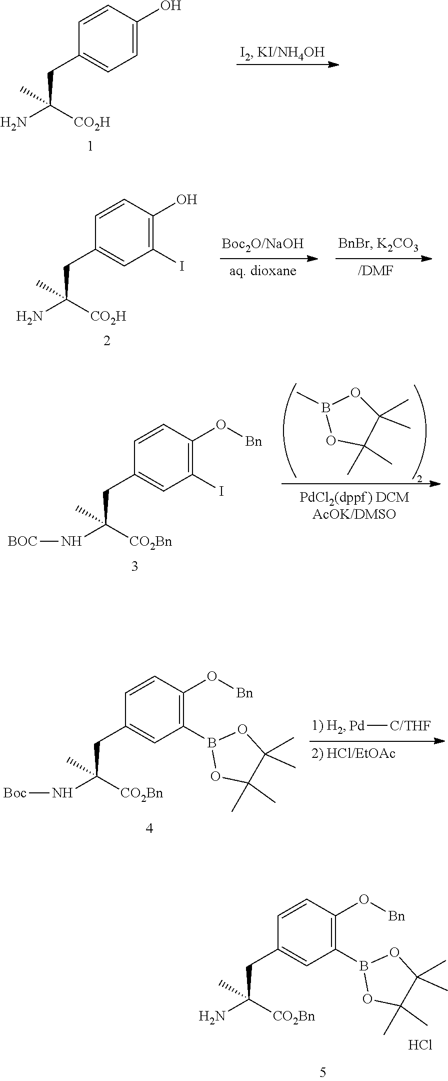

a) 3-iodo-α-methyl-L-tyrosine (2)

To a mixed solution of potassium iodide (10.2 g, 61.4 mmol) and water (27 mL) was added iodine (13.6 g, 53.6 mmol), and the mixture was stirred for 2 hr. Then, the mixture was added at −5° C. or lower to a mixed solution cooled to −7° C. of α-methyl-L-tyrosine (1) (10 g, 51.2 mmol), conc. aqueous ammonia (28%, 120 mL) and water (15 mL), and the mixture was stirred at −7 to −5° C. for 2 hr. To the reaction solution was added 15% aqueous sodium sulfite solution (25 mL), and the mixture was allowed to warm to room temperature, and concentrated under reduced pressure. The pH of the residual solution was adjusted to 6.5 to 7 with 6M hydrochloric acid under ice-cooled, the mixture was stirred under ice-cooled for 1 hr, and the precipitated solid was collected by filtration. The solid was washed with cold water and acetone, and dried under reduced pressure to give 3-iodo-α-methyl-L-tyrosine (2) (14.2 g, yield 86%).

¹H-NMR (300 MHz, DMSO-d₆+TFA, TMS): 8.25(3H, br), 7.51 (1H, d, J=2.1 Hz), 7.03 (1H, dd, J=2.1, 8.1 Hz), 6.84 (1H, d, J=8.1 Hz), 3.03 (1H, d, J=14.1 Hz), 2.87 (1H, d, J=14.1 Hz), 1.44 (3H, s)

b) N-Boc-3-iodo-O-benzyl-α-methyl-L-tyrosine benzyl ester (3)

To a mixed solution of 3-iodo-α-methyl-L-tyrosine (2) (3.0 g, 9.3 mmol), 1M aqueous sodium hydroxide solution (9.3 mL) and 1,4-dioxane (15 mL) was added di-t-butyl dicarbonate (4.06 g, 18.6 mmol), and the mixture was stirred at 50° C. for 24 hr. Additional di-t-butyl dicarbonate (2.0 g, 9.2 mmol) was added thereto, and the mixture was again stirred at 60° C. for 20 hr. The reaction solution was allowed to cool to room temperature, and concentrated under reduced pressure. The residue was subjected to separation extraction with water (30 mL) and methyl t-butyl ether-heptane (1:2, 45 mL). The organic layer was washed with 5% aqueous sodium hydrogencarbonate solution (20 mL) and water (20 mL). The pH of the combined washings was adjusted to 1-2 with 0.5M aqueous sodium hydrogen sulfate solution, and the mixture was subjected to extraction with methyl t-butyl ether-ethyl acetate (1:1, 60 mL). The extract was washed with water and saturated brine. The solvent was evaporated under reduced pressure to give a white solid (2.88 g). The obtained white solid (2.88 g) was dissolved in N,N-dimethylformamide (20 mL), potassium carbonate (2.08 g, 15 mmol) and benzyl bromide (1.8 mL, 15 mmol) were added thereto, and the mixture was stirred at 50° C. for 3 hr. The mixture was allowed to cool to room temperature, and ethyl acetate (50 mL) was added thereto. The mixture was washed with water and saturated brine, and the solvent was evaporated under reduced pressure. The residue was purified by silica gel column chromatography to give N-Boc-3-iodo-O-benzyl-α-methyl-L-tyrosine benzyl ester (3) as a white solid (3.39 g, yield 61%). ¹H-NMR (300 MHz, CDCl₃, TMS): 7.6-7.3 (11H, m), 6.88 (1H, dd, J=2.1, 8.4 Hz), 6.67 (1H, d, J=8.4 Hz), 5.20 (1H, d, J=12.6 Hz), 5.14 (1H, d, J=12.6 Hz), 5.10 (2H, s), 3.30 (1H, d, J=13.5 Hz), 3.15 (1H, d, J=13.5 Hz), 1.55 (3H, s), 1.48 (9H, s)

c) N-Boc-3-(4,4,5,5-tetramethyl-1,3,2-dioxaboran-2-yl)-O-benzyl-α-methyl-L-tyrosine benzyl ester (4)

Under nitrogen atmosphere, to a mixed solution of N-Boc-3-iodo-O-benzyl-α-methyl-L-tyrosine benzyl ester (3) (3.05 g, 5.0 mmol), [1,1′-bis(diphenylphosphino)ferrocene]dichloropalladium(II) dichloromethane adduct (0.31 g, 0.38 mmol), potassium acetate (1.96 g, 20 mmol) and dimethyl sulfoxide (20 mL) was added bis(pinacolato)diboron (2.54 g, 10 mmol), and the mixture was stirred at room temperature for 1 hr, and then stirred at 55° C. for 2 hr. The reaction solution was allowed to cool to room temperature, methyl t-butyl ether (45 mL) and water (45 mL) were added thereto, and the mixture was filtered through Celite. After liquid separation, the aqueous layer was subjected to extraction with methyl t-butyl ether (30 mL), and the organic layers were combined, and washed with water, half-saturated brine and saturated brine, and the solvent was evaporated under reduced pressure. The residue was purified by silica gel column chromatography to give N-Boc-3-(4,4,5,5-tetramethyl-1,3,2-dioxaboran-2-yl)-O-benzyl-α-methyl-L-tyrosine benzyl ester (4) as a white amorphous solid (2.22 g, yield 74%).

¹H-NMR (300 MHz, CDCl₃, TMS): 7.7-7.0 (12H, m) , 6.79 (1H, d, J=8.4 Hz), 5.3-5.0 (3H, m), 5.08 (2H, s), 3.31 (1H, d, J=13.8 Hz), 3.18 (1H, d, J=13.8 Hz), 1.46 (9H, s), 1.33 (12H, s), 1.26 (3H, s)

d) 3-(4,4,5,5-tetramethyl-1,3,2-dioxaboran-2-yl)-α-methyl-L-tyrosine hydrochloride (5)

A mixed solution of N-Boc-3-(4,4,5,5-tetramethyl-1,3,2-dioxaboran-2-yl)-O-benzyl-α-methyl-L-tyrosine benzyl ester (4) (2.2 g, 3.6 mmol), 10% palladium-carbon (55% hydrous, 0.55 g) and tetrahydrofuran (22 mL) was stirred under hydrogen gas atmosphere for 3 hr. The reaction solution was filtered through Celite, and the solvent was evaporated under reduced pressure to give a white amorphous solid (1.69 g). To a mixed solution of the obtained amorphous solid (0.56 g, 1.2 mmol) and ethyl acetate (1.5 mL) was added 4M hydrochloric acid-ethyl acetate solution (1.5 mL), and the mixture was stirred at room temperature for 1 hr. The precipitated solid was collected by filtration, and washed with ethyl acetate to give 3-(4,4,5,5-tetramethyl-1,3,2-dioxaboran-2-yl)-α-methyl-L-tyrosine hydrochloride (5) as a white powder (0.30 g, yield 70%). ¹H-NMR (300 MHz, DMSO-d₆, TMS): 8.87 (1H, s), 8.35 (3H, br), 7.33 (1H, d, J=2.4 Hz), 7.15 (1H, dd, J=2.4, 8.4 Hz), 6.79 (1H, d, J=8.4 Hz), 3.04 (1H, d, J=14.1 Hz), 2.95 (1H, d, J=14.1 Hz), 1.45 (3H, s), 1.26 (12H, s)

### HPLC Analysis Condition

sample solution: 3-(4,4,5,5-Tetramethyl-1,3,2-dioxaboran-2-yl)-α-methyl-L-tyrosine hydrochloride (5) (1 mg) was dissolved in water containing 67% acetonitrile (1 mL) to give a sample solution.
detector: UV 275 nm
column: YMC-Pack Pro C18 RS (4.6 mmφ×25 cm, 5 μm, YMC)
column temperature: 30° C.
mobile phase: H₂O (0.1% TFA)/MeCN (0.1% TFA)
mode: gradient
   0 min H₂O (0.1% TFA)/MeCN (0.1% TFA)=95/5
   5 min H₂O (0.1% TFA)/MeCN (0.1% TFA)=95/5
   35 min H₂O (0.1% TFA)/MeCN (0.1% TFA)=5/95
   40 min H₂O (0.1% TFA)/MeCN (0.1% TFA)=5/95
flow rate: 1 mL/min
injection volume: 5 μL
analysis time: 40 min
retention time: 3-(4,4,5,5-tetramethyl-1,3,2-dioxaboran-2-yl)-α-methyl-L-tyrosine hydrochloride (5): 12.3 min

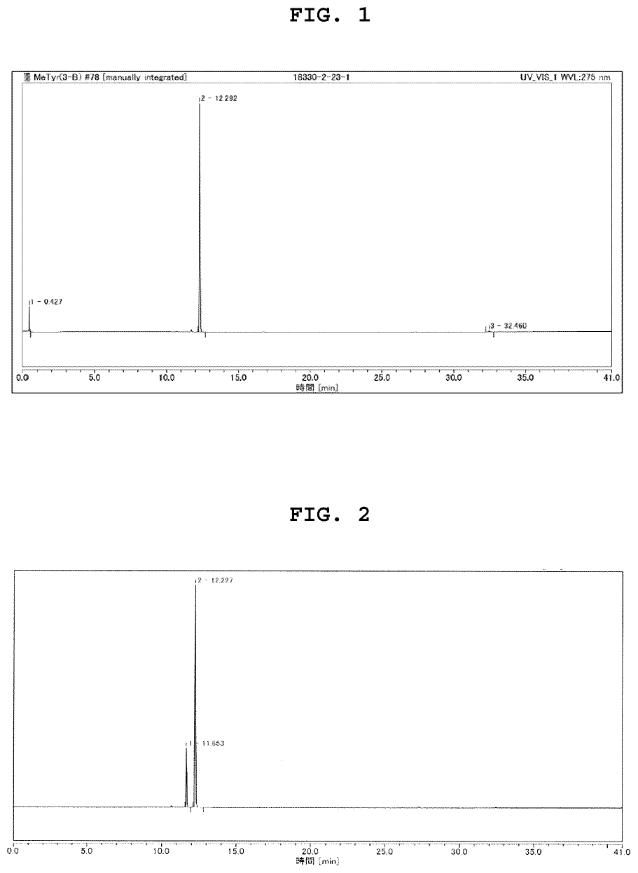

The HPLC chart is shown in FIG. 1.

### Example 2

### Synthesis of 3-borono-α-methyl-L-tyrosine hydrochloride (5)

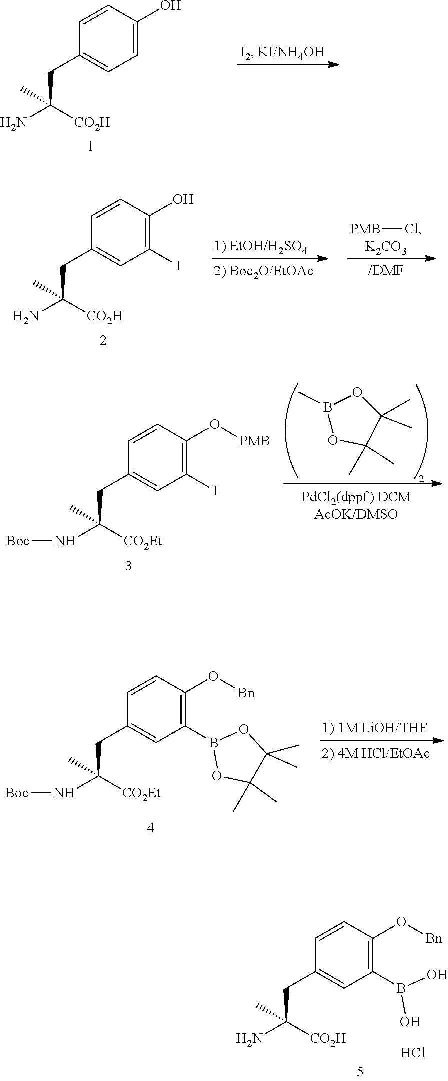

a) 3-iodo-α-methyl-L-tyrosine (2)

To a mixed solution of potassium iodide (10.2 g, 61.4 mmol) and water (27 mL) was added iodine (13.6 g, 53.6 mmol), and the mixture was stirred for 2 hr. The mixture was added at −5° C. or lower to a mixed solution cooled to −7° C. of α-methyl-L-tyrosine (1) (10 g, 51.2 mmol), conc. aqueous ammonia (28%, 120 mL) and water (15 mL), and stirred at −7-−5° C. for 2 hr. To the reaction solution was added 15% aqueous sodium sulfite solution (25 mL), and the mixture was allowed to warm to room temperature, and concentrated under reduced pressure. The pH of the residue solution was adjusted to 6.5 to 7 with 6M hydrochloric acid under ice-cooled, and the mixture was stirred under ice-cooled for 1 hr. The precipitated solid was collected by filtration, washed with cold water and acetone, and dried under reduced pressure to give 3-iodo-α-methyl-L-tyrosine (2) (14.2 g, yield 86%).

¹H-NMR (300 MHz, DMSO-d₆+TFA, TMS): 8.25(3H, br), 7.51 (1H, d, J=2.1 Hz), 7.03 (1H, dd, J=2.1, 8.1 Hz), 6.84 (1H, d, J=8.1 Hz), 3.03 (1H, d, J=14.1 Hz), 2.87 (1H, d, J=14.1 Hz), 1.44 (3H, s)

b) N-Boc-3-iodo-O-(p-methoxybenzyl)-α-methyl-L-tyrosine ethyl ester (3)

To a mixed solution of 3-iodo-α-methyl-L-tyrosine (2) (3.0 g, 9.3 mmol) and ethanol (75 mL) was added conc. sulfuric acid (1.0 mL, 18.6 mmol), and the mixture was stirred for 48 hr while heating under reflux. The reaction solution was allowed to cool to room temperature, and concentrated. To the residue were added ice water (30 mL) and sodium hydrogencarbonate (3.9 g), and the mixture was subjected to extraction with ethyl acetate (50 mL). The extract was washed with 5% aqueous sodium hydrogencarbonate solution, water and saturated brine, and the solvent was evaporated under reduced pressure to give a pale-yellow syrup (1.7 g). The obtained residue was dissolved in ethyl acetate (17 mL), and di-t-butyl dicarbonate (1.1 g, mmol) was added thereto under ice-cooled, and the mixture was stirred at room temperature for 2 hr. Additional di-t-butyl dicarbonate (1.1 g, 5 mmol) was added thereto under ice-cooled, and the mixture was stirred at room temperature for 20 hr. The solvent of the reaction solution was evaporated under reduced pressure, and the residue was purified by silica gel column chromatography to give N-Boc-3-iodo-α-methyl-L-tyrosine ethyl ester (Intermediate A) as a white solid (2.0 g). The obtained white solid (1.85 g, 4.1 mmol) was dissolved in N,N-dimethylformamide (10 mL), potassium carbonate (0.68 g, 4.9 mmol) and p-methoxybenzyl chloride (0.6 mL, 4.4 mmol) were added thereto, and the mixture was stirred at 50° C. for 2 hr. The mixture was allowed to cool to room temperature, ethyl acetate (50 mL) was added thereto, and the mixture was washed with water and saturated brine. The solvent was evaporated under reduced pressure, and the residue was purified by silica gel column chromatography to give N-Boc-3-iodo-O-(p-methoxybenzyl)-α-methyl-L-tyrosine ethyl ester (3) as a white solid (2.24 g, yield 46%, 3 step).

N-Boc-3-iodo-α-methyl-L-tyrosine ethyl ester (Intermediate A) ¹H-NMR (300 MHz, CDCl₃, TMS): 7.39 (1H, d, J=2.1 Hz), 6.95 (1H, dd, J=2.1, 8.4 Hz), 6.87 (1H, d, J=8.4 Hz), 5.28 (1H, br), 5.19(1H, br), 4.23 (2H, q, J=7.2 Hz), 3.33 (1H, d, J=13.5 Hz), 3.12 (1H, d, J=13.5 Hz), 1.61 (3H, s), 1.49 (9H, s), 1.31 (3H, t, J=7.2Hz)

N-Boc-3-iodo-O-(p-methoxybenzyl)-α-methyl-L-tyrosine ethyl ester (3)

¹H-NMR (300 MHz, CDCl₃, TMS): 7.52 (1H, d, J=2.4 Hz), 7.40 (2H, d, J=8.7 Hz), 6.98 (1H, dd, J=2.4, 8.4 Hz), 6.90 (2H, d, J=8.7 Hz), 6.75 (1H, d, J=8.4 Hz), 5.18 (1H, br), 5.04 (2H, s), 4.21 (2H, m), 3.82 (3H, s), 3.32 (1H, d, J=13.5 Hz), 3.11 (1H, d, J=13.5 Hz), 1.58 (3H, s), 1.49 (9H, s), 1.31 (3H, t, J=6.9 Hz)

c) N-Boc-3-(4,4,5,5-tetramethyl-1,3,2-dioxaboran-2-yl)-O-(p-methoxybenzyl)-α-methyl-L-tyrosine ethyl ester (4)

Under nitrogen atmosphere, to a mixed solution of N-Boc-3-iodo-O-(p-methoxybenzyl)-α-methyl-L-tyrosine ethyl ester (3) (2.6 g, 4.57 mmol), [1,1′-bis(diphenylphosphino)ferrocene]dichloropalladium(II) dichloromethane adduct (0.22 g, 0.27 mmol), potassium acetate (1.8 g, 18.3 mmol) and dimethyl sulfoxide (20 mL) was added bis(pinacolato)diboron (2.32 g, 9.14 mmol), and the mixture was stirred at room temperature for 1 hr, and then stirred at 55° C. for 4 hr. The reaction solution was allowed to cool to room temperature, and methyl t-butyl ether (60 mL) and half-saturated brine (60 mL) were added thereto. The mixture was filtered through Celite, and the filtrate was subjected to liquid separation. The organic layer was washed with half-saturated brine, 5% aqueous sodium hydrogencarbonate solution and saturated brine, and the solvent was evaporated under reduced pressure. The residue was purified by silica gel column chromatography to give N-Boc-3-(4,4,5,5-tetramethyl-1,3,2-dioxaboran-2-yl)-O-(p-methoxybenzyl)-α-methyl-L-tyrosine ethyl ester (4) as a white solid (1.66 g, yield 64%). ¹H-NMR (300 MHz, CDCl₃, TMS): 7.50 (2H, d, J=8.7 Hz), 7.40 (1H, d, J=2.7 Hz), 7.11(1H, dd, J=2.7, 8.4 Hz), 6.89 (2H, d, J=8.7 Hz), 6.83 (1H, d, J=8.4 Hz), 5.18 (1H, br), 5.02 (2H, s), 4.20 (2H, q, J=7.5 Hz), 3.82 (3H, s), 3.32 (1H, d, J=13.2 Hz), 3.13(1H, d, J=13.2 Hz), 1.60 (3H, s), 1.48 (9H, s), 1.33 (12H, s), 1.29 (3H, t, J=7.5 Hz)

d) 3-borono-α-methyl-L-tyrosine hydrochloride (5)

To a mixed solution of N-Boc-3-(4,4,5,5-tetramethyl-1,3,2-dioxaboran-2-yl)-O-(p-methoxybenzyl)-α-methyl-L-tyrosine ethyl ester (4) (1.3 g, 2.28 mmol), tetrahydrofuran (19.5 mL) and water (6.5 mL) was added lithium hydroxide monohydrate (0.43 g, 10.2 mmol), and the mixture was stirred at room temperature. The reaction solution was concentrated under reduced pressure, and the residue was diluted with water (25 mL). 0.5M Aqueous sodium hydrogen sulfate solution (30 mL) was added thereto (pH 2-3), and the mixture was subjected to extraction with MTBE (30 mL). The extract was washed with water and saturated brine, and the solvent was evaporated under reduced pressure to give a white solid (1.02 g). To the obtained solid was added 4M hydrochloric acid-ethyl acetate solution, the mixture was stirred at room temperature for 30 min, and the solvent was evaporated under reduced pressure. The residue was washed with ethyl acetate, and dried to give crude 3-borono-α-methyl-L-tyrosine hydrochloride (5) (0.42 g) as a white powder. This powder (0.40 g) was dissolved in a small amount of H₂O, and subjected to preparative purification with ODS separation column under conditions of mobile phase: 0.1% HCl aq./acetonitrile and gradient: 100/0 to 95/5. The objective fractions were collected, and the solvent was evaporated. The residue was dissolved in 0.1% HCl aq., and subjected to cotton-plug filtration. The solvent was evaporated under reduced pressure, and the residue was dried under reduced pressure by vacuum pump to give 3-borono-α-methyl-L-tyrosine hydrochloride (5) (283 mg, yield 45%). ¹H-NMR (300 MHz, DMSO-d₆+5% D₂O, TMS): 8.42 (1H, s), 7.46 (1H, m, J=2.4 Hz), 7. 00-7.17 (1H, m), 6.72-6.82 (1H, m), 3.06 (1H, d, J=14.1 Hz), 2.95 (1H, d, J=14.1 Hz), 1.47 (3H, s)

### HPLC Analysis Condition

sample solution: 3-Borono-α-methyl-L-tyrosine hydrochloride (5) (1 mg) was dissolved in 0.1% HCl aq. (1 mL) to give a sample solution.
detector: UV 275 nm
column: YMC-Pack Pro C18 RS (4.6 mmφ×25 cm, 5 μm, YMC)
column temperature: 30° C.
mobile phase: H₂O (0.1% TFA)/MeCN (0.1% TFA)
mode: gradient
   0 min H₂O (0.1% TFA)/MeCN (0.1% TFA)=95/5
   5 min H₂O (0.1% TFA)/MeCN (0.1% TFA)=95/5
   35 min H₂O (0.1% TFA)/MeCN (0.1% TFA)=5/95
   40 min H₂O (0.1% TFA)/MeCN (0.1% TFA)=5/95
flow rate: 1 mL/min
injection volume: 5 μL
analysis time: 40 min
retention time: 3-borono-α-methyl-L-tyrosine hydrochloride (5): 11.7 min, 12.2 min (detected as a mixture of the boroxine (associated molecule) and the monomer)

The HPLC chart is shown in FIG. 2.

### Reference Example 1

### Preparation of ²¹¹At Aqueous Solution

According to the method described in Reference Example 1 of Patent Document 2, ²¹¹At aqueous solution was prepared. That is, ²¹¹At was produced by ²⁰⁹Bi(α,2n) ²¹¹At nuclear reaction resulting from the irradiation of bismuth with helium particles accelerated (28 MeV) by a cyclotron. After the irradiation, by heating, the target substance ²⁰⁹Bi was melted, while the ²¹¹At was vaporized. The vaporized ²¹¹At was collected to a cooled trap, and dissolved in a small amount of water to prepare an ²¹¹At aqueous solution.

### Example 3

### Synthesis of 3-astato (²¹¹At)-α-methyl-L-tyrosine

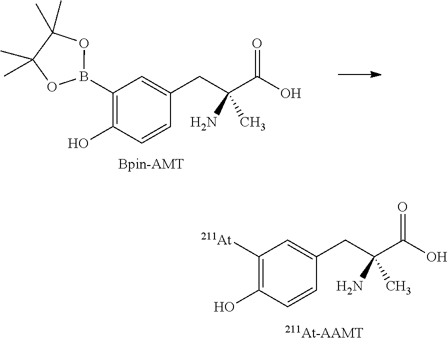

3-(4,4,5,5-Tetramethyl-1,3,2-dioxaboran-2-yl)-α-methyl-L-tyrosine hydrochloride (Bpin-AMT) was dissolved in water to prepare a 0.8 w/v % aqueous solution. This aqueous solution (0.1 mL) was put in a microtube, and then the ²¹¹At aqueous solution (0.02 mL, 5 MBq) prepared in Reference Example 1 and 0.1M aqueous KI solution (0.05 mL) were added thereto, and the mixture was reacted at 50° C. for 1 hr to give a crude product solution of 3-astato (²¹¹At)-α-methyl-L-tyrosine (²¹¹At-AAMT).

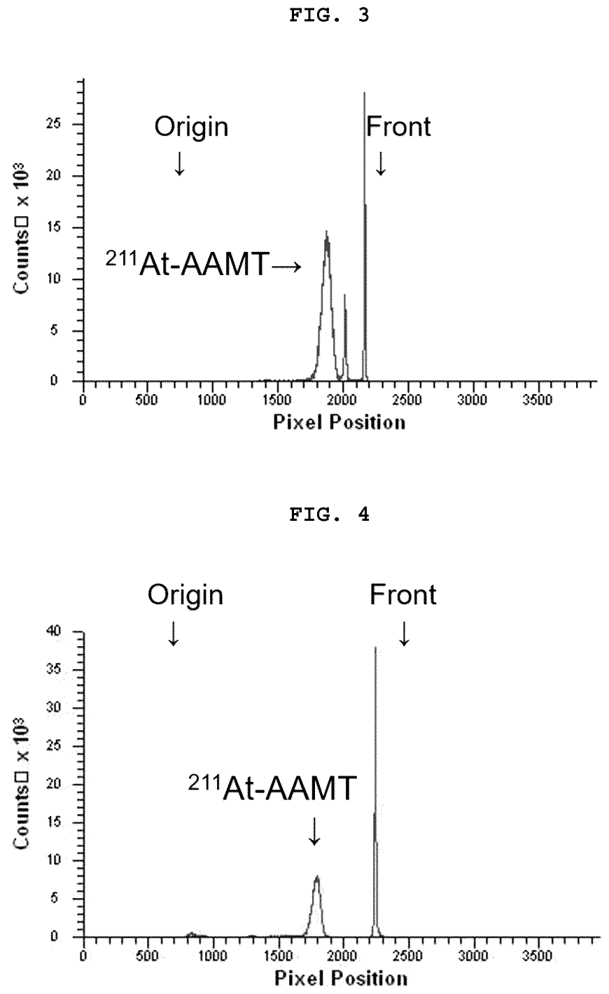

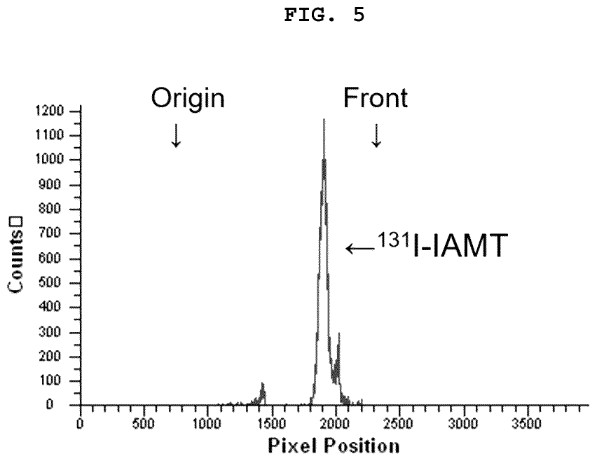

This crude product solution (1 μL) was analyzed by thin-layer chromatograph method (TLC). The sample was spotted on a thin-layer plate (silica gel G60), and a mixed solvent of acetonitrile/water (2/1) was used as a developing solvent. The thin-layer plate was exposed to an imaging plate for about 15 min, and then analyzed by a bioimage analyzer (Typhoon FLT7000, GE Healthcare, Chicago). The results of the TLC analysis are shown in FIG. 3. 3-Astato (²¹¹At)-α-methyl-L-tyrosine in the crude product solution was detected at Rf 0.78, and the radiochemical yield (RCY) was 74.9%.

The crude product solution was injected into an Oasis HLB cartridge (Waters) to capture the target product, and water (1 mL) was passed through the cartridge to elute impurities. Next, 30% ethanol solution (1 mL) was passed through the cartridge to elute the target product to give the purified product. The radiochemical yield and radiochemical purity of the purified product were 74% and 94.8%, respectively.

### Example 4

### Synthesis of 3-astato (²¹¹At)-α-methyl-L-tyrosine

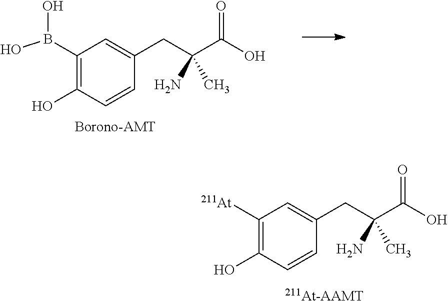

3-Borono-α-methyl-L-tyrosine hydrochloride (Borono-AMT) was dissolved in water to prepare a 1 w/v % aqueous solution. This aqueous solution (0.1 mL) was put in a microtube, and then the ²¹¹At aqueous solution (0.02 mL, 5 MBq) prepared in Reference Example 1 and 0.1M aqueous KI solution (0.02 mL) were added thereto, and the mixture was reacted at 50° C. for 1 hr to give a crude product solution of 3-astato (²¹¹At)-α-methyl-L-tyrosine (²¹¹At-AAMT).

This crude product solution (1 μL) was analyzed by thin-layer chromatograph method (TLC). The sample was spotted on a thin-layer plate (silica gel G60), and a mixed solvent of acetonitrile/water (2/1) was used as a developing solvent. The thin-layer plate was exposed to an imaging plate for about 15 min, and then analyzed by a bioimage analyzer (Typhoon FLT7000, GE Healthcare, Chicago). The results of the TLC analysis are shown in FIG. 4. 3-Astato (²¹¹At)-α-methyl-L-tyrosine was detected at Rf 0.78, and the radiochemical yield (RCY) was 68.2%.

The crude product solution was injected into an Oasis HLB cartridge to capture the target product, and water (1 mL) was passed through the cartridge to elute impurities. Next, 30% ethanol solution (1 mL) was passed through the cartridge to elute the target product to give the purified product. The radiochemical yield and radiochemical purity of the purified product were 58.3% and 90.9%, respectively.

### Example 5

### Synthesis of 3-iodo(¹³¹I)-α-methyl-L-tyrosine

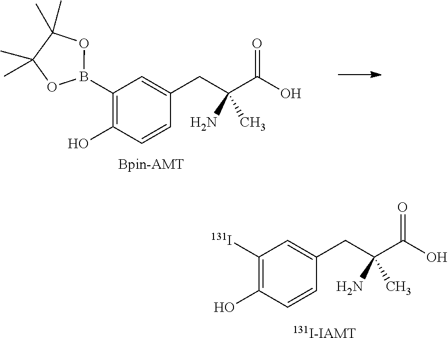

3-(4,4,5,5-Tetramethyl-1,3,2-dioxaboran-2-yl)-α-methyl-L-tyrosine hydrochloride (Bpin-AMT) was dissolved in water to prepare a 1 w/v % aqueous solution. This aqueous solution (0.1 mL) was put in a microtube, and then aqueous [¹³¹I]sodium iodide solution (0.03 mL, 1 MBq, Institute of Isotopes Co., Ltd.) and 0.4 w/v% aqueous N-bromosuccinimide (NBS) solution (0.03 mL) were added thereto, and the mixture was reacted at room temperature for 30 min. Then, to the reaction solution was added 3 w/v% aqueous ascorbic acid solution (0.03 mL) to stop the reaction to give a crude product solution of 3-iodo(¹³¹I)-α-methyl-L-tyrosine (¹³¹I-IAMT).

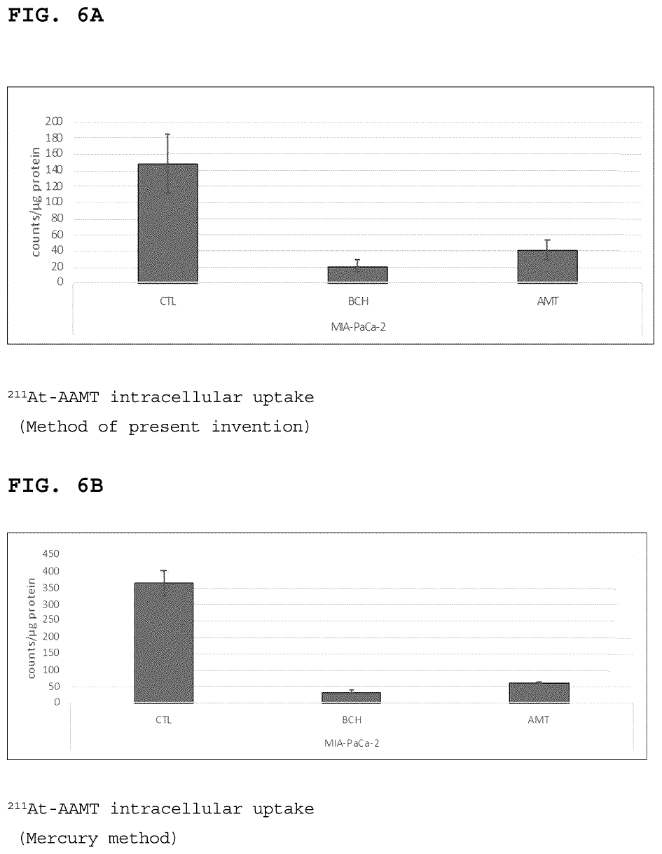

This crude product solution (1 μL) was analyzed by thin-layer chromatograph method (TLC). The sample was spotted on a thin-layer plate (silica gel G60), and a mixed solvent of acetonitrile/water (2/1) was used as a developing solvent. The thin-layer plate was exposed to an imaging plate for about 15 min, and then analyzed by a bioimage analyzer (Typhoon FLT7000, GE Healthcare, Chicago). The results of the TLC analysis are shown in FIG. 5. 3-Iodo(¹³¹I)-α-methyl-L-tyrosine was detected at Rf 0.72, and the radiochemical yield (RCY) was 86.1%. This compound was stable up to 7 days after labeling.

### Reference Example 2

### Synthesis of 3-astato (²¹¹At)-α-methyl-L-tyrosine

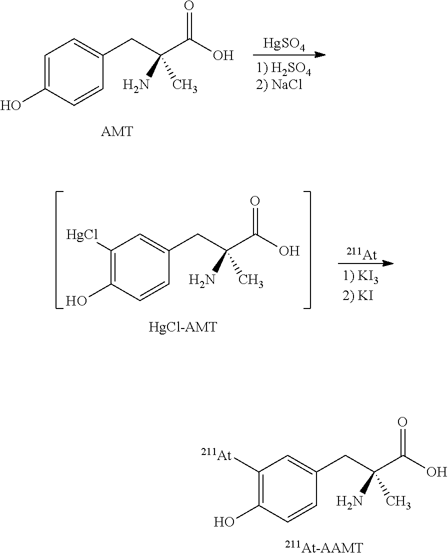

α-Methyl-L-tyrosine (AMT) (22 μmol) and HgSO₄ (20 μmol) were added to H₂SO₄ aqueous solution (0.2 M, 0.5 mL), and the mixture was stirred at room temperature for 2 hr. NaCl (45 μmol) was added to the reaction solution, and the mixture was stirred for 5 min. To the reaction solution were added the ²¹¹At aqueous solution (about 5 MBq/mL, 100 μL) prepared in Reference Example 1 and 1M KI₃ (5 μL), and the mixture was stirred for 30 min. Then, an appropriate amount (100 μL or more) of 1M KI solution was added thereto until the suspension became clear to stop the reaction. The obtained reaction solution was desalted through a cation column (Dowex™ 50W×8 100-200 mesh, 0.2M aqueous NH₃ solution), followed by an anion column (Dowex™ 1×8 50-100 mesh, 2% aqueous AcOH solution) to give 3-astato (²¹¹At)-α-methyl-L-tyrosine (²¹¹At-AAMT) Furthermore, sodium ascorbate was added thereto to a final concentration of 1 w/v % for stabilization. The yield was 60 to 80%.

### Example 6

This example demonstrates that the ²¹¹At-AAMT synthesized by the method of the present invention is taken up by cancer cells via an amino acid transporter (LAT1) that is specifically expressed in cancer cells, and that this performance is comparable to that of the ²¹¹At-AAMT synthesized by the conventional mercury method.

Human pancreatic cancer cells MIA PaCa-2 were seeded in a 24-well culture plate at a concentration of 1×10⁵ cells/mL. After 2 days, the medium was removed, the cells were washed with PBS (−), and the medium was replaced with HEPES Buffer (amino acid free). To each well, the ²¹¹At-AAMT synthesized by the method of the present invention or the conventional mercury method was added. In addition, an excess amount (1 mM) of BCH (LAT1 inhibitor) or unlabeled (AMT) was added. After culturing the cells for 30 minutes, the cells were washed with PBS (−), and lysed with 0.1 N NaOH, and the radioactivity of the lysate was measured. The ²¹¹At-AAMT intracellular uptake (radioactivity count/protein amount) was calculated by correcting the radioactivity per protein amount. The results are shown in FIG. 6 (FIG. 6a: the method of the present invention, FIG. 6b: the conventional mercury method). In FIG. 6, CTL is control, BCH is 2-aminobicyclo[2,2,1]heptane-2-carboxylic acid, and AMT is α-methyltyrosine. The ²¹¹At-AAMT synthesized by the method of the present invention was strongly taken up by pancreatic cancer cells (CTL), and the uptake was significantly inhibited in the presence of BCH and AMT. This result was consistent with that of the ²¹¹At-AAMT synthesized by conventional techniques. From these results, it was confirmed that the ²¹¹At-AAMT synthesized by the method of the present invention is specifically taken up into pancreatic cancer cells via LAT1.

### Example 7

This example demonstrates that the ²¹¹At-AAMT synthesized by the method of the present invention has cytotoxicity against cancer cells, and that this performance is comparable to that of the ²¹¹At-AAMT synthesized by the conventional mercury method.

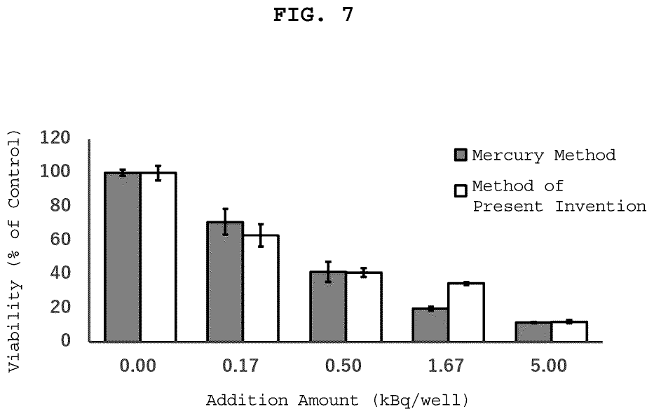

HEK293 cells were seeded in a 24-well culture plate at a concentration of 1×10⁵ cells/mL. After 2 days, the medium was removed, the cells were washed with PBS (−), and the medium was replaced with HEPES Buffer (amino acid free). To each well, 0.00-5.00 kBq of the ²¹¹At-AAMT synthesized by the method of the present invention or the conventional mercury method was added. After culturing the cells for 24 hours, the cell viability was examined. The results are shown in FIG. 7. In the group of the ²¹¹At-AAMT synthesized by the method of the present invention, the cell viability decreased in a dose-dependent manner. This result was consistent with that of the group of the ²¹¹At-AAMT synthesized by the conventional mercury method. From these results, it was confirmed that the ²¹¹At-AAMT synthesized by the method of the present invention has the same cytotoxicity as that of the conventional mercury method. Industrial Applicability

According to the present invention, tyrosine derivatives into which a boryl group (—B(OH)₂) or its ester group is introduced can be obtained, and therefore, the use of the compounds makes it possible to produce radiolabeled tyrosine derivatives with good purity and stability, by a safe method suitable for industrial production of pharmaceuticals without using hazardous substances.

This application is based on patent application No. 2021-052352 filed on Mar. 25, 2021 in Japan, the contents of which are encompassed in full herein.

1. A method for producing a compound represented by Formula (5) or a salt thereof, comprising the following Steps 1 to 4; 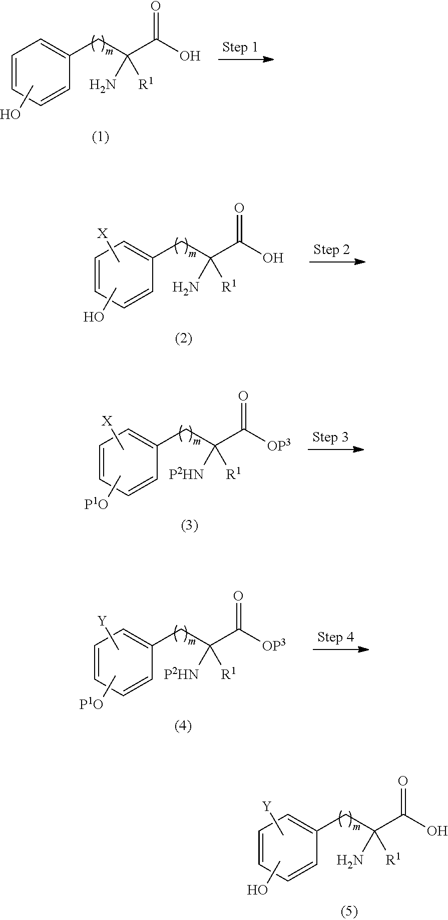
wherein
R¹ is a hydrogen atom or a C₁₋₄ alkyl group;
P¹ is an ether-type hydroxy-protecting group;
P² is an amino-protecting group;
P³ is a carboxy-protecting group;
m is 0, 1 or 2;
X is a halogen atom; and
Y is a boryl group (—B(OH)₂) or its ester group,
Step 1: a step of halogenating a compound represented by Formula (1) or a salt thereof to obtain a compound represented by Formula (2) or a salt thereof;
Step 2: a step of protecting the amino group and carboxy group of the compound represented by Formula (2) or a salt thereof and protecting the hydroxy group of the compound or salt with an ether-type protecting group to obtain a compound represented by Formula (3);
Step 3: a step of reacting the compound represented by Formula (3) with a reagent for introducing boronic acid in the presence of a palladium catalyst and a base to obtain a compound represented by Formula (4); and
Step 4: a step of removing the protecting groups for the carboxy group, amino group and hydroxy group of the compound represented by Formula (4) to obtain the compound represented by Formula (5) or a salt thereof.

2. The method according to claim 1, wherein P1 is a benzyl group or a p-methoxybenzyl group.

3. The method according to claim 1, wherein P³ is a benzyl group or a C₁₋₂ alkyl group.

4. The method according to claim 1, wherein P¹ and P³ are both benzyl groups.

5. The method according to claim 1, wherein the bonding position of the hydroxy group on the benzene ring in Formula (5) is the 4-position or 3-position.

6. The method according to claim 5, wherein the bonding positions of the hydroxy group and the group −Y on the benzene ring in Formula (5) are adjacent to each other.

7. The method according to claim 1, wherein, in Formula (5), the bonding position of the hydroxy group on the benzene ring is the 4-position, and the bonding position of the group −Y on the benzene ring is the 3-position.

8. The method according to claim 1, wherein R¹ is a hydrogen atom or a methyl group.

9. The method according to claim 1, wherein the palladium catalyst to be used in Step 3 is [1,1′-bis(diphenylphosphino)ferrocene]dichloropalladium(II) (PdCl₂(dppf)).

10. The method according to claim 9, wherein the reaction of Step 3 is carried out in a sulfoxide solvent or an amide solvent.

11. The method according to claim 9, wherein the base to be used in Step 3 is an alkali metal acetate.

12. The method according to claim 1, wherein Y is a boryl group (—B(OH)₂) or a 4,4,5,5-tetramethyl-1,3,2-dioxaboran-2-yl group.

13. A method for producing a compound represented by Formula (5) or a salt thereof, comprising the following Step 4; 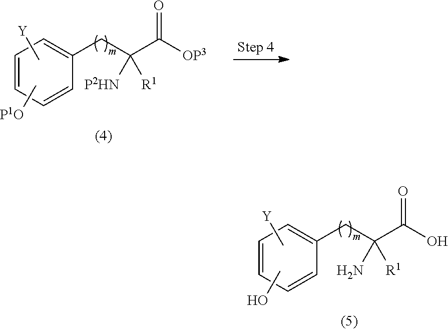
wherein
R¹ is a hydrogen atom or a C₁₋₄ alkyl group;
P¹ is an ether-type hydroxy-protecting group;
P² is an amino-protecting group;
P³ is a carboxy-protecting group;
m is 0, 1 or 2; and
Y is a boryl group (—B(OH)₂) or its ester group,
Step 4: a step of removing the protecting groups for the carboxy group, amino group and hydroxy group of a compound represented by Formula (4) to obtain the compound represented by Formula (5) or a salt thereof.

14. A method for producing a compound represented by Formula (4), comprising the following Step 3; 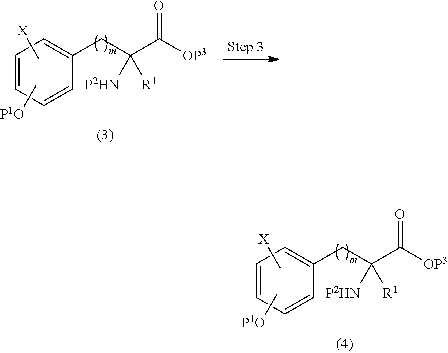
wherein
R¹ is a hydrogen atom or a C₁₋₄ alkyl group;
P¹ is an ether-type hydroxy-protecting group;
P² is an amino-protecting group;
P³ is a carboxy-protecting group;
m is 0, 1 or 2;
X is a halogen atom; and
Y is a boryl group (—B(OH)₂) or its ester group,
Step 3: a step of reacting a compound represented by Formula (3) with a reagent for introducing boronic acid in the presence of a palladium catalyst and a base to obtain the compound represented by Formula (4).

15. A compound represented by the following Formula (5a) or a salt thereof; 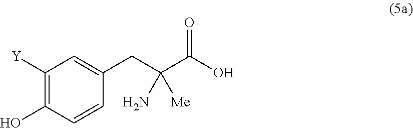
wherein Y is a boryl group (—B(OH)₂) or its ester group.

16. A compound represented by the following Formula (4a); 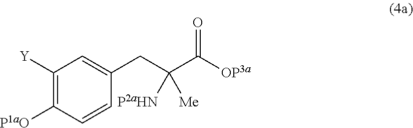
wherein
P^{1a} is a benzyl group or a p-methoxybenzyl group;
P^{2a} is a tert-butoxycarbonyl group;
P^{3a} is a benzyl group or a C₁₋₂ alkyl group; and
Y is a boryl group (—B(OH)₂) or its ester group.

17. A method for producing a radiolabeled compound represented by Formula (6) or a salt thereof, comprising the following Step 5; 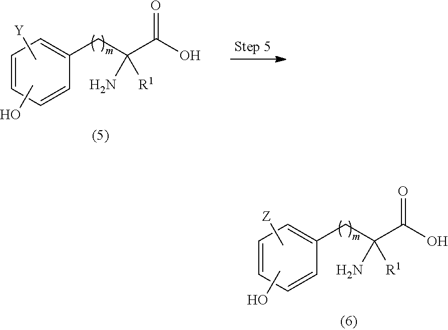
wherein
R¹ is a hydrogen atom or a C₁₋₄ alkyl group;
m is 0, 1 or 2;
Y is a boryl group (—B(OH)₂) or its ester group; and
Z is ²¹¹At, ²¹⁰At, ¹²³I, ¹²⁴I, ¹²⁵I or ¹³¹I,
Step 5: a step of reacting a compound represented by Formula (5) or a salt thereof with a radionuclide selected from ²¹¹At, ²¹⁰At, ¹²³I, ¹²⁴I, ¹²⁵I and ¹³¹I, in the presence of a reagent selected from an alkali metal iodide, an alkali metal bromide, N-bromosuccinimide, N-chlorosuccinimide, N-iodosuccinimide and hydrogen peroxide, in water to obtain the radiolabeled compound represented by Formula (6) or a salt thereof.

18. The method according to claim 17, wherein the compound represented by Formula (5) or a salt thereof is produced by a method comprising the following Steps 1 to 4: 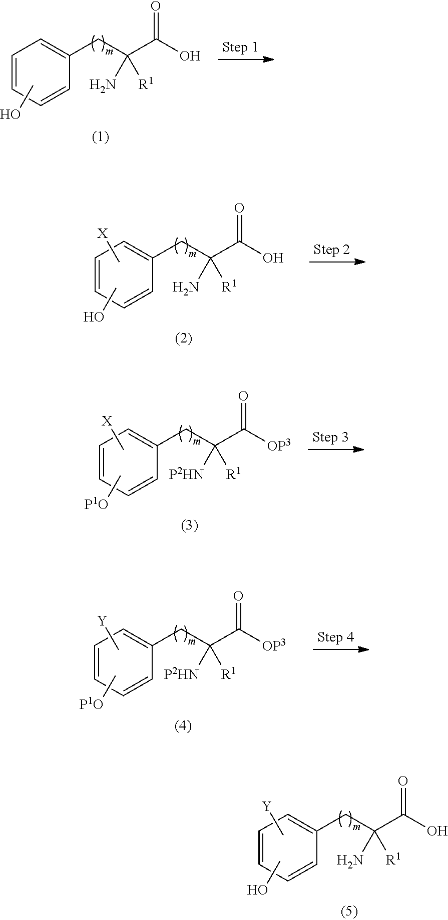
wherein
R¹ is a hydrogen atom or a C₁₋₄ alkyl group;
P¹ is an ether-type hydroxy-protecting group;
P² is an amino-protecting group;
P³ is a carboxy-protecting group;
m is 0, 1 or 2;
X is a halogen atom; and
Y is a boryl group (—B(OH)₂) or its ester group,
Step 1: a step of halogenating a compound represented by Formula (1) or a salt thereof to obtain a compound represented by Formula (2) or a salt thereof;
Step 2: a step of protecting the amino group and carboxy group of the compound represented by Formula (2) or a salt thereof and protecting the hydroxy group of the compound or salt with an ether-type protecting group to obtain a compound represented by Formula (3);
Step 3: a step of reacting the compound represented by Formula (3) with a reagent for introducing boronic acid in the presence of a palladium catalyst and a base to obtain a compound represented by Formula (4); and
Step 4: a step of removing the protecting groups for the carboxy group, amino group and hydroxy group of the compound represented by Formula (4) to obtain the compound represented by Formula (5) or a salt thereof.

19. The method according to claim 17, wherein the reaction is carried out in an organic solvent-free system.

20. The method according to claim 17, wherein the reaction is carried out within the range of room temperature to 100° C.

21. The method according to claim 17, wherein the radionuclide is ²¹¹At or ¹³¹I, and the reagent is selected from potassium iodide and N-bromosuccinimide.

22. The method according to claim 17, further comprising a step of purifying the radiolabeled compound represented by Formula (6) or a salt thereof.

23. The method according to claim 17, further comprising a step of stabilizing the radiolabeled compound represented by Formula (6) or a salt thereof by adding ascorbic acid.
